(19) United States
(12) Patent Application Publication
(10) Pub. No.: US 2013/0018309 A1
(43) Pub. Date: Jan. 17, 2013

(54) TEAR-AWAY SHEATH WITH INTEGRAL STOP

(71) Applicants: Benjamin T. Ewing, Wexford, PA (US); Scott A. Bednar, Freedom, PA (US); Brian P. Dickerson, Mount Plesant, PA (US); Steven D. Savage, Paynesville, MN (US); Gregory G. Brucker, Minneapolis, MN (US)

(72) Inventors: Benjamin T. Ewing, Wexford, PA (US); Scott A. Bednar, Freedom, PA (US); Brian P. Dickerson, Mount Plesant, PA (US); Steven D. Savage, Paynesville, MN (US); Gregory G. Brucker, Minneapolis, MN (US)

(21) Appl. No.: 13/516,003

(22) PCT Filed: Dec. 20, 2010
(86) PCT No.: PCT/US2010/061317

§ 371 Date: Sep. 24, 2012

Related U.S. Application Data
(60) Provisional application No. 61/287,824, filed on Dec. 18, 2009.

(51) Int. Cl. A61M 25/01; A61M 25/10

(52) U.S. Cl. 604/103.05; 604/171

(57) ABSTRACT

A tear-away sheath (8) having a tubular element (10) with a distal end and a proximal end, at least one axial fault line (12), and an integral stop (16) is disclosed. The tear-away sheath may also have a handle (14) or closing element at the proximal end. An angioplasty balloon catheter (22) having the disclosed tear-away sheath is also disclosed.

### FIELD OF THE DISCLOSURE

Embodiments of the present disclosure generally relate to a tear-away sheath for use with medical devices, for example therapeutic coated balloon catheters.

### BACKGROUND

The following background information is provided to assist the reader to understand embodiments disclosed herein and the environment in which they may be used. The terms used herein are not intended to be limited to any particular narrow interpretation unless clearly stated otherwise, either expressly or impliedly, in this document.

Sheaths placed over catheter balloons serve two main functions: 1) to provide a protective barrier against mechanical damage and contamination by foreign material; and 2) to constrain expansion of the balloon during manufacturing and storage to keep balloon profiles as low as possible for crossing tight lesions in the vascular system. For example, the sheath is an extruded tube made of a lubricious material, such as Teflon® polymers, that is placed over the balloon after its folding is completed. The sheath fits snugly over the balloon and remains in place until the balloon is prepared for clinical use.

During the manufacturing process of a coated balloon catheter, the existing sheath on the uncoated balloon must be removed for the application of the coating. Once coated, anew, larger sheath must be used to constrain the balloon since it gained thickness through the addition of the coating.

Therapeutic coated catheter balloons are particularly delicate. Such therapeutic coatings are generally friable and easily removed from the balloon with slight pressure such as touching or contact with hard surfaces, for example the preparation table. Premature release of the therapeutic coating outside the patient exposes medical personnel to therapeutic particles and reduces therapeutic delivery to the target site. As such, there is a need for sheaths that protect a therapeutic coated catheter balloon until it has been introduced into the vascular system.

During use, removal of the sheath is best performed as the balloon is being inserted into the patient. To ensure a complete transfer of the therapeutic agent, the sheath is typically inserted into an introducer containing a hemostasis valve and then the balloon is pushed into the vessel of the patient. If the sheath were not inserted into the introducer, then the hemostasis valve could scrape away significant portions of the therapeutic coating as it was being inserted into the patient. By inserting the sheath through the hemostasis valve, the coated balloon is protected until it is pushed into the vessel of the patient. To remove the sheath from the inserted balloon catheter, a peel-away or tear-away sheath having a line of weakness or slit must be used. However, a need exists to control the depth of the sheath insertion during this process for ease of use by the practitioner and to avoid having the sheath penetrate too far into the vessel.

### SUMMARY

In general, various embodiments of the present disclosure are directed to a tear-away sheath with integral stop for medical devices, particularly therapeutic coated balloon catheters. The tear-away sheath may remain on the device until after the device, such as a balloon catheter, has entered the vascular system.

In one embodiment, the tear-away sheath comprises a tubular element with a distal end and a proximal end, at least one axial fault line, and a stop. The axial fault line may be a slit, a notch, a slot, a score line, a perforated zipper, a thinner section extruded into the tubular element, or a characteristic of a plastic used to form the tubular element. The stop may also have at least one axial fault line aligned with the at least one axial fault line of the tubular element. The tear-away sheath may have a handle at the proximal end of the tubular element for assisting with splitting the tear-away sheath. In alternate embodiments, the tear-away sheath may have a closing band, a pull-tab, at least one tab with a corresponding slot, or an opening with a wing at the proximal end for removing the tear-away sheath. The tear-away sheath may also be split using a cutting device, either external or integrated on the medical device.

In another embodiment, an angioplasty balloon catheter having the disclosed tear-away sheath is disclosed. Methods of inserting a balloon catheter into the vascular system using a tear-away sheath embodied herein are also disclosed. Further embodiments relate to methods of forming a tear-away sheath with a stop.

These and other details, objects, and advantages of the present disclosure will become better understood or apparent from the following description and drawings showing embodiments thereof.

### BRIEF DESCRIPTION OF THE DRAWINGS

The accompanying drawings illustrate examples of embodiments of the disclosure. In such drawings:

FIG. 1 shows embodiments of a tear-away sheath without an integral stop (A), with an integral stop (B), and various examples of handles (B-H) embodied herein;

FIG. 2 is a cross-section of embodiments of the tubular element of a tear-away sheath with a slit (A) and a notch (B);

FIG. 3 demonstrates a cross-section of a tear-away sheath on a balloon catheter positioned in an introducer;

FIG. 4 shows embodiments of a tear-away sheath having a stop extending the length of the tubular element (A) and a cross-section thereof (B);

FIG. 5 shows embodiments of the tubular element of a tear-away sheath inside a positioning block and a heating element for forming the stop;

FIG. 6 demonstrates embodiments of slits in the distal end of the tubular element (A) for forming the stop (B) by pushing back;

FIG. 7 shows embodiments of a tear-away sheath having a graduated larger outside diameter that forms the stop;

FIG. 8 shows embodiments of a tear-away sheath with a closing band and a helical fault line;

FIG. 9 shows embodiments of a tear-away sheath with a pull-tab and an axial straight fault line in a closed position (A) and an open position (B);

FIG. 10 shows embodiments of a tear-away sheath with a tab and corresponding slot closing feature in an open position (A) and a closed position (B);

FIG. 11 shows embodiments of the proximal end of a catheter with a cutting device integrated on the hub;

FIG. 12 demonstrates embodiments of splitting a tear-away sheath using an extension on the proximal end of the catheter shaft (A), on the strain relief (B), or integrated on the hub of the catheter (C);

FIG. 13 demonstrates embodiments of cutting the tubular element with a stop (A) at a bent elbow near the proximal end (B) to form an opening for insertion of a balloon catheter and a wing for removing the tear-away sheath from the catheter (C);

FIG. 14 is a cross-section of embodiments of a tear-away sheath with an O-ring and a retaining feature forming the stop embodied herein;

FIG. 15 is a cross-section of embodiments of a tear-away sheath with an O-ring and an end cap inserted into the distal end of the tubular element forming the stop embodied herein; and

FIG. 16 demonstrates embodiments of a tear-away sheath having a stop-tab and an axial fault line integrated into the tubular element (A) and inserted into an introducer (B).

### DESCRIPTION

In all of its embodiments and related aspects, the present disclosure may be used with medical devices, including, for example, angioplasty balloon catheters. Other examples of medical devices include, without limitation, drainage catheters, ventricular catheters, ventriculostomy balloons, balloon expandable stents, coronary balloons, drug eluting stents, drug eluting balloons, any of the above with therapeutic coatings, or combination devices with therapeutic coatings, and the like.

Medical devices are routinely coated with compositions including, for example and without limitation, therapeutic agents, radiopaque materials, radioactive materials, polymeric materials, sugars, waxes, fats, and lubricious materials. As used herein, “therapeutic agent” or “therapeutic coating” includes, but is not limited to, any therapeutic, for example drugs, genetic material, and biological material. Genetic material includes for example, without limitation, DNA or RNA, viral vectors and non-viral vectors. Biological material includes for example, without limitation, cells, bacteria, proteins such as growth factors, peptides, lipids, and hormones. Drugs include for example, without limitation, anti-thrombogenic agents, anti-proliferative agents, anti-inflammatory agents, anti-neoplastic agents such as epothilone and its derivatives, antimiotic agents, antioxidants, anti-coagulants, immunosuppressants such as limus drugs (e.g. sirolimus (rapamycin), biolimus, everolimus, tacrolimus, zotarolimus) and their derivatives, vascular cell growth promoters, vascular cell growth inhibitors, antibiotic agents, angiogenic substances, restenosis-inhibiting agents, and drugs for heart failure. The “therapeutic agent” may include a combination of one or more therapeutics. Particular embodiments include restenosis-inhibiting agents such as rapamycin, mitomycin C, Taxol, paclitaxel, paclitaxel analogues, derivatives, and mixtures thereof. The coatings can be in solid, liquid, or gas forms depending on the method used to coat the device. In an example, carriers may be used with the therapeutic, such as, for example and without limitation, bioabsorbable agents, microspheres, microtubes, and physiologically compatible non-reactive drug transfer or radio opaque agents, including but not limited to urea, iopromide and other iodine or gadolinium based contrast agents, cremophore EL, vitamin E, Tocopheryl Polyethylene Glycol Succinate (TPGS), shellac, surfactants, and the like.

Various embodiments described herein pertain to a tear-away sheath for medical devices, particularly angioplasty balloon catheters. In general, a balloon catheter has a shaft with a distal end, a proximal end, and at least one lumen, a balloon on the distal end, and a strain relief and a hub at the proximal end. The disclosed tear-away sheath may be particularly useful for protecting and inserting a catheter balloon that has a specialized coating containing a therapeutic agent, i.e. a therapeutic coating.

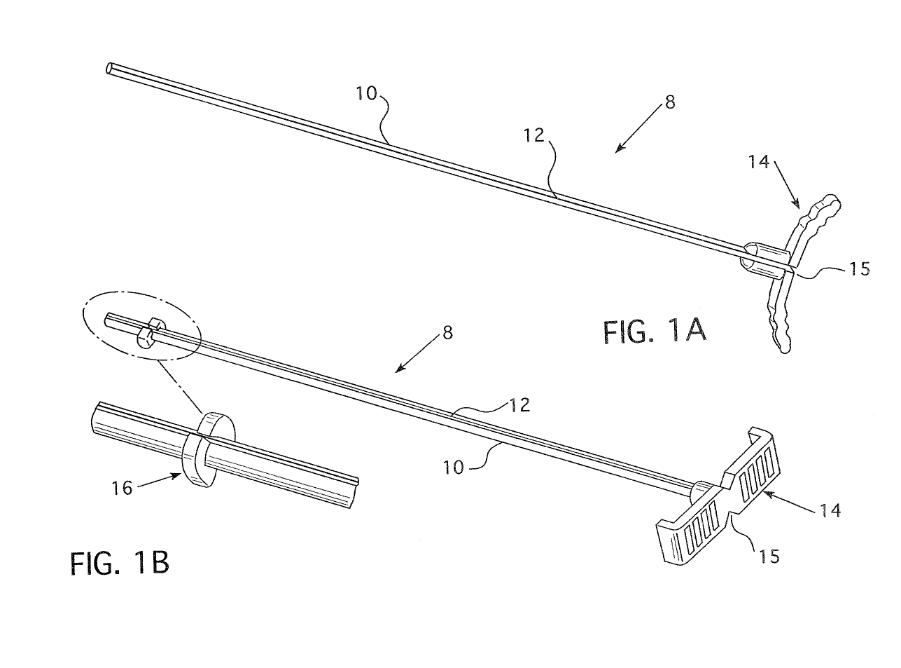

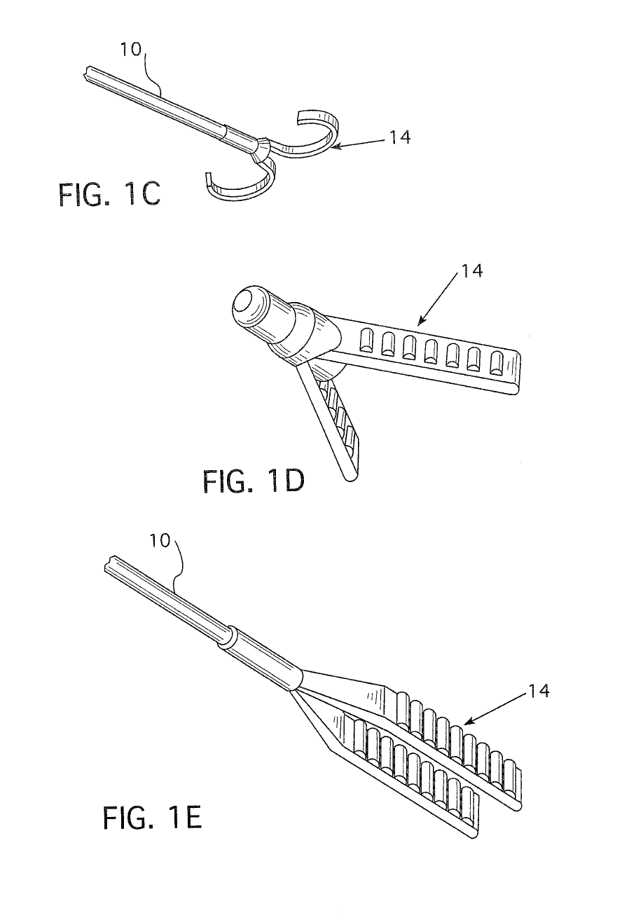

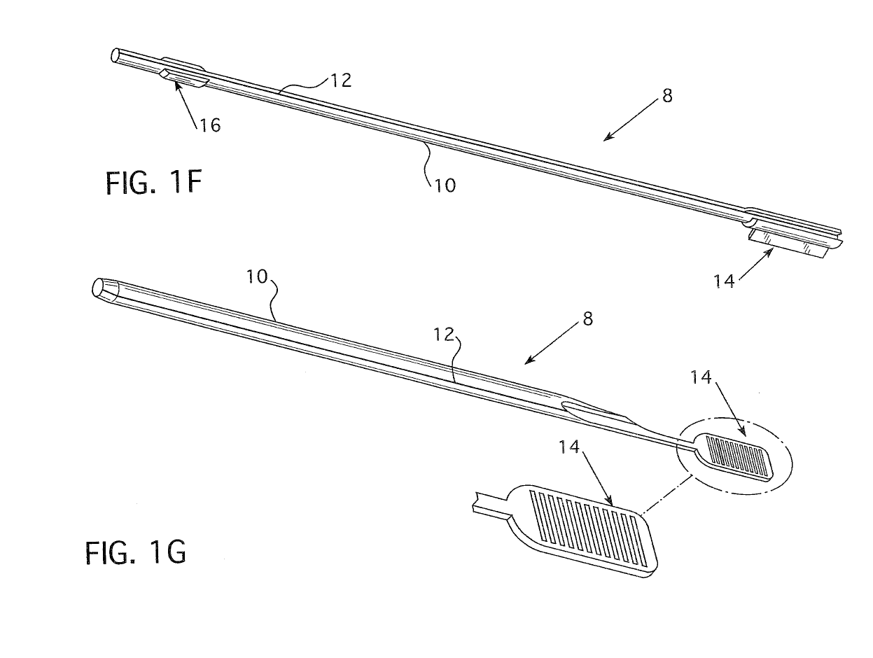

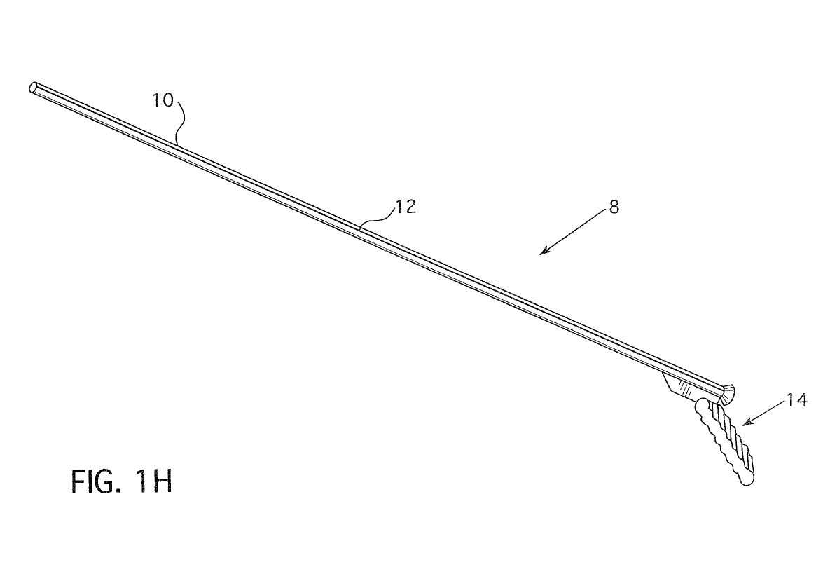

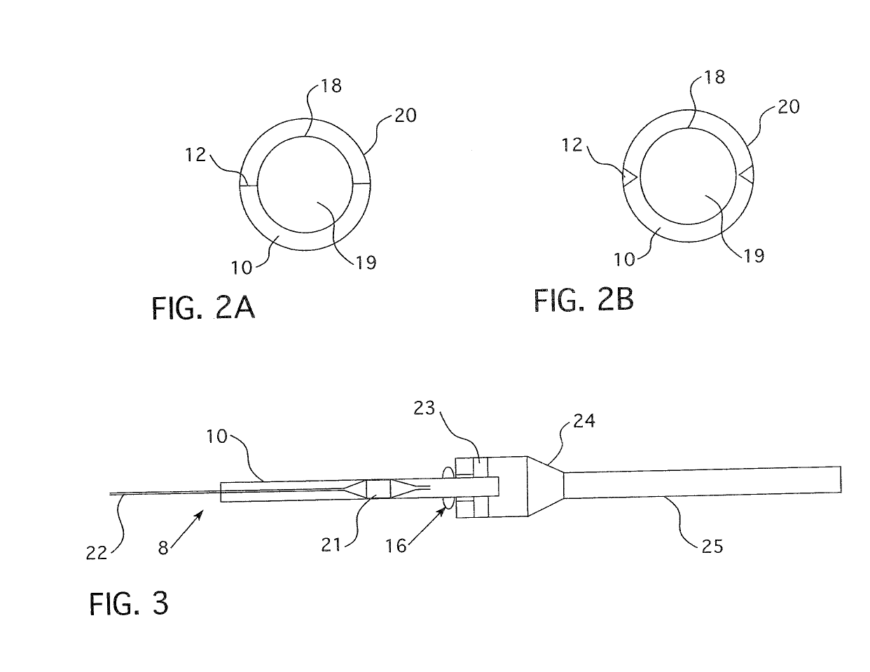

In one embodiment, the tear-away sheath 8 has a tubular element 10 with a distal end and a proximal end, at least one axial fault line 12, and a handle 14 at the proximal end, i.e. closest to a user. See FIG. 1A. The tear-away sheath 8 may also have a stop 16, for example, near the distal end of the tubular element 10. See FIG. 1B. The tubular element 10 may be made of a plastic that is extrudable and/or moldable, for example without limitation, Teflon® polymers (e.g. PTFE, PFA, ETFE, FEP), polyethylenes (e.g. PE, HDPE), polypropolyene (PP), nylon, polyester, polyvinylchloride (PVC), etc. In an example, the tubular element 10 is extruded from PTFE or FEP. The selection of plastic material for the tubular element 10 may have several requirements. First, in one embodiment, the plastic is hard enough to provide the requisite axial stiffness for insertion of a catheter balloon 21 through the lumen 19 of the tubular element 10 and into the vascular system. See FIGS. 2 and 3. In another embodiment, the plastic tears easily and smoothly either as a characteristic of the plastic, e.g. axial orientation of the polymeric chains, or along preformed axial fault lines 12. For example, PTFE, FEP, and other Teflon® polymers will tear linearly without an axial fault line 12. In one embodiment, the plastic is lubricious enough to allow for easy passage of a coated balloon 21 through the lumen 19 of the tubular element 10. See FIGS. 2 and 3. The plastic may be capable of being molded, e.g. a nylon or polyester, or compatible to other plastics to be applied via insert molding and/or other means of bonding, for example and without limitation ultrasonic welding, infrared (IR) welding, adhesive or chemical bonding, laser welding, and the like, to attach the handle 14 and the stop 16.

The inner diameter 18 of the tubular element 10 may be sized to provide proper clearance over a coated balloon 21 as the balloon 21 is inserted into the lumen 19. See FIGS. 2 and 3. In one embodiment, the clearance is sufficient to constrain the diameter of the balloon 21 from expanding to maintain the smallest profile possible and also provide a frictional fit to prevent movement or loss of the tear-away sheath 8 during handling and transportation while at the same time protecting the balloon 21. For purposes of illustration only, the coated balloon 21 appears without folds in FIG. 3 in order to demonstrate the relation of the tear-away sheath 8 and the introducer 24. Those of skill in the art will understand that the coated balloon 21 in its folded state inside of the tear-away sheath 8 has a profile that is not much larger than that of catheter 22. In an example, the inner diameter 18 of the tubular element 10 is about 0.001 to about 0.040 inches larger than the sheath used on an uncoated balloon 21. This increase in the inner diameter 18 of the lumen 19 is necessary since a therapeutic coated balloon 21 is thicker than an uncoated balloon 21. In an example, the diameter of the lumen 19 for the tear-away sheath 8 of the present disclosure ranges from about 0.004 to about 0.008 inches larger than the sheath used for the uncoated balloon 21. Since different sized balloons 21 have their own particular delivery sheaths to maintain the tight fit described above, which vary in length and diameter, the tear-away sheath 8 of embodiments of the present invention may be similarly varied in size, yet with a larger lumen 19 diameter as described herein. The outer diameter 20 may be sized to provide sufficient axial stiffness so that the longest balloons 21 can be inserted through the tear-away sheath 8 by holding the handle 14 and pushing along the shaft of the catheter 22. For example, a wall thickness that varies depending on the tear-away sheath 8 material and length, may be from about 0.001 to about 0.1 inches with a diameter of the lumen 19 from about 0.008 to about 0.040 inches, in order to provide sufficient axial stiffness. In one embodiment, the length of the tubular element 10 is sufficient to cover the entire catheter balloon 21 with at least about ≧1 cm extra on each side of the balloon 21. The tear-away sheath 8 may cover just the balloon 21 or up to the entire catheter 22 or other device.

The tubular element 10 may have at least one preferential axial fault line 12. See FIGS. 1A and 1B. In an example, the tubular element 10 has a fault line 12 on diametrically opposite sides. See FIG. 2. The fault line 12 may be any device or feature for assisting in tearing away the sheath 8, such as, for example without limitation, a slit (shown in FIG. 2A), slot, or notch (shown in FIG. 2B) that extends at least partially through the wall thickness of the tubular element 10, a thinner section extruded into the tubular element 10, a score line, a perforated zipper, or may be a characteristic of the plastic, e.g. axial orientation of the polymeric chains. The axial fault line may extend from the outside surface inward or from the inside surface outward.

At the proximal end, the tubular element 10 may interface with a handle 14. See FIGS. 1A-H. The handle 14 holds the tubular element 10 of the tear-away sheath 8 and may initiate the tearing process. The handle 14 may be designed to be held easily with one hand to keep the tear-away sheath 8 inserted into an introducer 24 and to maintain proper orientation to the introducer 24 for easy insertion of the catheter balloon 21. See FIG. 3. The handle 14 may also have at least one notch 15 that aligns with the axial fault line 12 of the tubular element 10. See FIGS. 1A and 1B. In an example, the handle 14 is winged shaped with a wing on each side of the notch 15 (shown in FIGS. 1A and B). Other examples of handle designs are shown in FIGS. 1C-E, which may allow for a more secure grip (shown in FIG. 1C), a single-handed grip (shown in FIG. 1D), or a compact design to minimize packaging and permit higher storage utilization (show in FIG. 1E). For example, when the handle 14 is snapped and pulled apart, the tear propagates along the fault line 12, thus separating the tear-away sheath 8 into two separate pieces. The plastic of the handle 14 may be, in various embodiments, the same as the tubular element 10. In an example, the material is thicker at the handle 14 to ensure that fracture occurs at the notch 15 and not at the handle 14. The plastic for the handle 14 may be a harder, more brittle plastic than the tubular element 10 and thus may fracture easily. The plastic may also bond to the plastic of the tubular element 10 or be adherent enough to permit the tearing of the tubular element 10. Examples include, without limitation, polycarbonates, acrylics, nylons, Teflon® polymers, polyethylenes, polypropolyene, polyester, and polyvinylchloride. The handle 14 may also be mechanically interlocked with the tubular element 10 during molding to provide adherence without a physical bond between the two plastic materials.

In another embodiment, the handle 14 may be multiple tabs positioned 90 degrees or approximately 90 degrees to the at least one axial fault line 12. See FIG. 1F. The tabs of the handle 14 may be molded to the tubular element 10 and protrude from the tubular element 10 to form an included angle of about 120 degrees. Such an angle may permit the tabs of the handle 10 to be grasped between an operator's thumb and forefinger and pinched together to open the axial fault line 12 and allow the catheter 22 shaft or other medical device to pass through the axial fault line 12.

In another embodiment, the handle 14 may be a single tab positioned or molded onto one side of the tubular element 10 diametrically opposite the axial fault line 12. See FIGS. 1G and H. FIG. 1G shows a section of the tubular element 10 removed from the axial fault line 12 side and the handle 14 configured as a tab molded onto the remaining material. FIG. 1H shows the handle 14 configured as a tab molded to the tubular element 10 opposite the axial fault line 12. In various embodiments, the handle 14 may be designed to fit between the thumb and forefingers or to be gripped with two hands and may utilize surface features to help secure the handle 14.

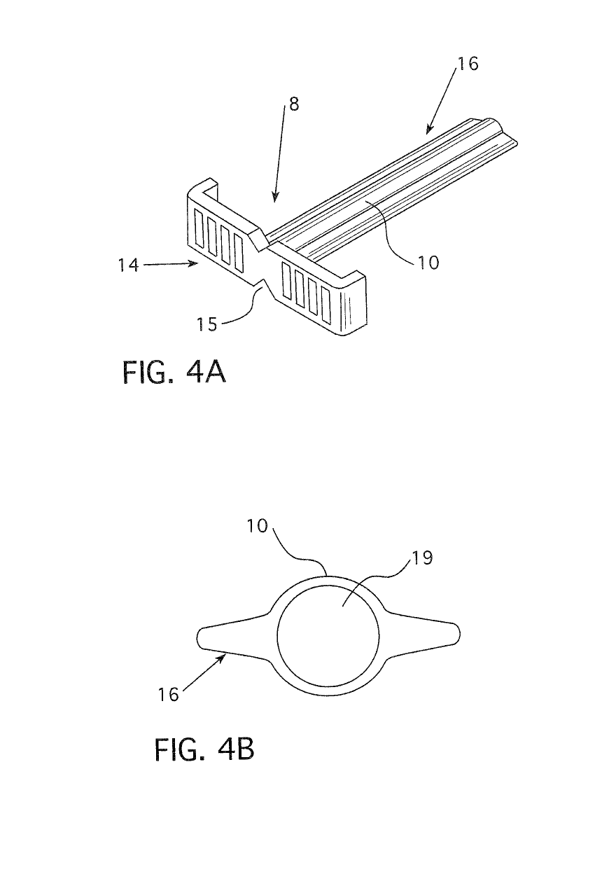

The tear-away sheath 8 may have a physical stop 16 for positioning the medical device, such as a balloon catheter 22. See FIGS. 1B and 3. For a balloon catheter 22, the stop 16 may be positioned such that the tubular element 10 distal to the stop 16 is inserted into and opens a flexible valve 23 housed within a hemostasis valve introducer 24, but the stop 16 prevents the tear-away sheath 8 from extending more distally into the introducer sheath 25 and entering the vessel. See FIG. 3. The stop 16 may be proximal from the distal end of the tubular element 10 such that the hemostasis valve 23 is penetrated by tubular element 10 but tear-away sheath 8 penetrates no further. In an example, the stop 16 may be at least about 0.04 inches proximal to the distal end of the tubular element 10. In an example, the position of the stop 16 can vary along the length of the tubular element 10 based on the size of the introducer 24, such as about 0.04 inches to about 13.78 inches proximal to the distal end of the tubular element 10. In another example, the stop 16 is about 0.400 inches from the distal end of the tubular element 10. In yet another example, the stop 16 may extend for the majority of the length of the tubular element 10. See FIGS. 4A and 4B. In this example, the stop 16 may be extruded the entire length of the tubular element 10 ending at a position proximal to the distal end of tubular element 10, such as within the ranges described above, such as at 0.04 inches proximal to the distal end of tubular element 10 (not shown). This configuration not only allows for the tear-away sheath 8 to enter the introducer 24 as described above, but also may increase the overall axial stiffness of tear-away sheath 8 which minimizes buckling of the tear-away sheath 8 while the balloon catheter 22 is being pushed into the vessel. This feature may be useful for tear-away sheaths 8 for smaller diameter and/or longer balloons 21. In another embodiment, the diameter of the stop 16 may range from about 0.125 to about 1.0 inch and may be designed to cover a broad range of introducer 24 sizes. In an example, the diameter of the stop 16 is about 0.250 inches. The stop 16 may also have at least one preferential axial fault line 12 aligned with the axial fault line(s) 12 of the tubular element 10 to permit continuous tearing for completely separating the tear-away sheath 8.

The present disclosure also relates to methods of forming a stop 16 on the tubular element 10 of the tear-away sheath 8. The stop 16 may be made of the same or a different material as the tubular element 10 for insert molding the stop 16 onto the tubular element 10. In one embodiment, the stop 16 may be bonded or molded onto the tubular element 10 near the distal end of the tubular element 10. For example, the stop 16 may be molded onto the distal end of the tubular element 10 in two locations at 90 degrees to the at least one axial fault line 12 to form two external features which become the stop 16. The stop may also be extruded with the tubular element 10, for example as shown in FIG. 4.

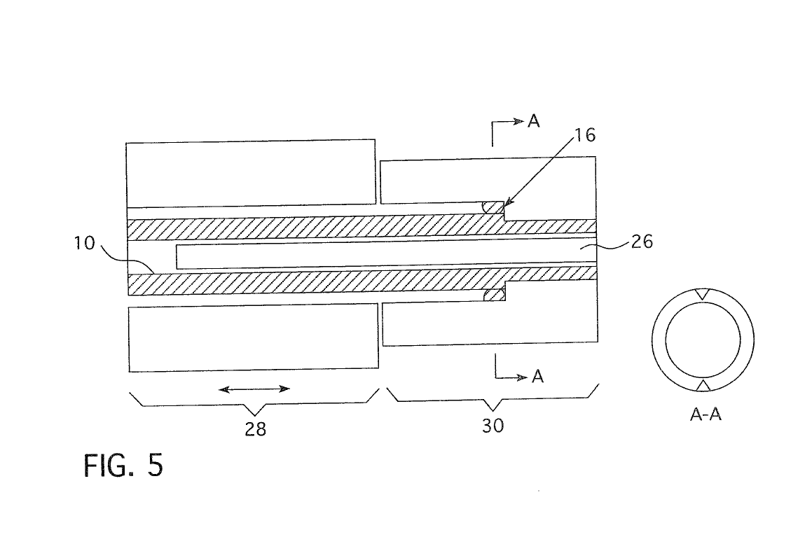

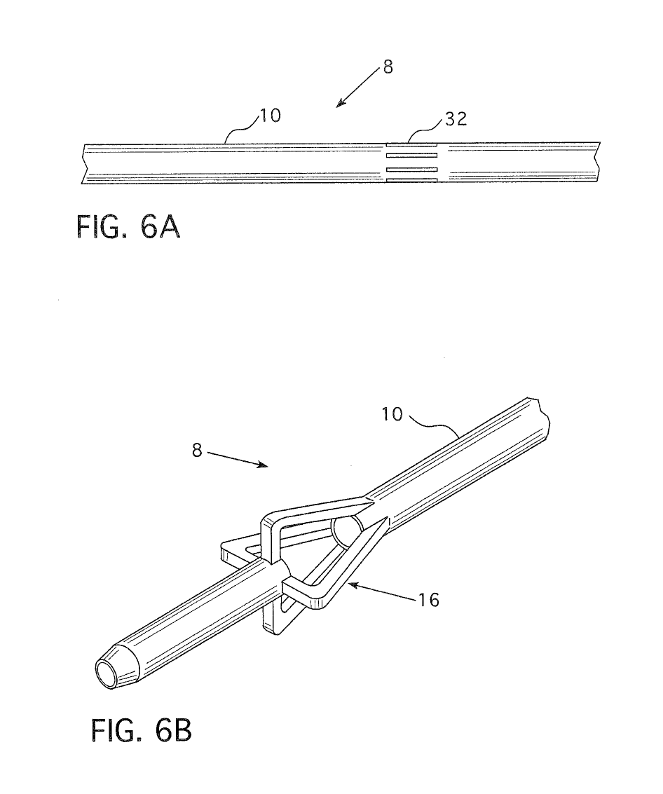

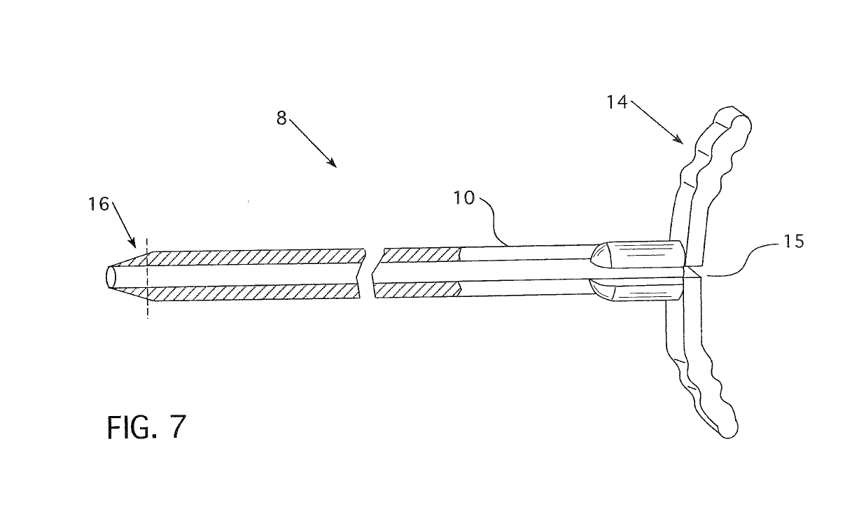

In the embodiment with the overall design depicted in FIG. 1, the stop 16 may be manufactured by loading the distal end of the tubular element 10 onto a pin gauge 26 with an outside diameter that closely matches the inside diameter of the tubular element 10. See FIG. 5. A sliding block fixture 28 surrounding the tubular element 10 is advanced forward, thus pushing the distal end of the tubular element 10 into a heated element 30 to form the stop 16. The inside diameter of the heated element 30 is smaller than the outside diameter of the tubular element 10, thus melting the wall of the tubular element 10 and pushing a ring of plastic backwards to form the stop 16. In another embodiment, slits 32 are formed or cut into the tubular element 10 near the distal end. See FIG. 6A. The distal end is then pushed back toward the proximal end of the tubular element 10, thereby expanding the slit area outward forming the stop 16. See FIG. 6B. In a further embodiment, the stop 16 may be formed by a graduated larger outer diameter along the length of the tubular element 10 preventing the tear-away sheath 8 from entering the introducer sheath 25. See FIG. 7. In another embodiment, the stop 16 may be fabricated onto the tubular element 10 with plastics that are moldable or not moldable. A mechanical feature may be required to interlock the stop 16 and the tubular element 10, for example and without limitation, a secondary molding process, ultrasonic welding, or adhesive bonding to the tubular element 10.

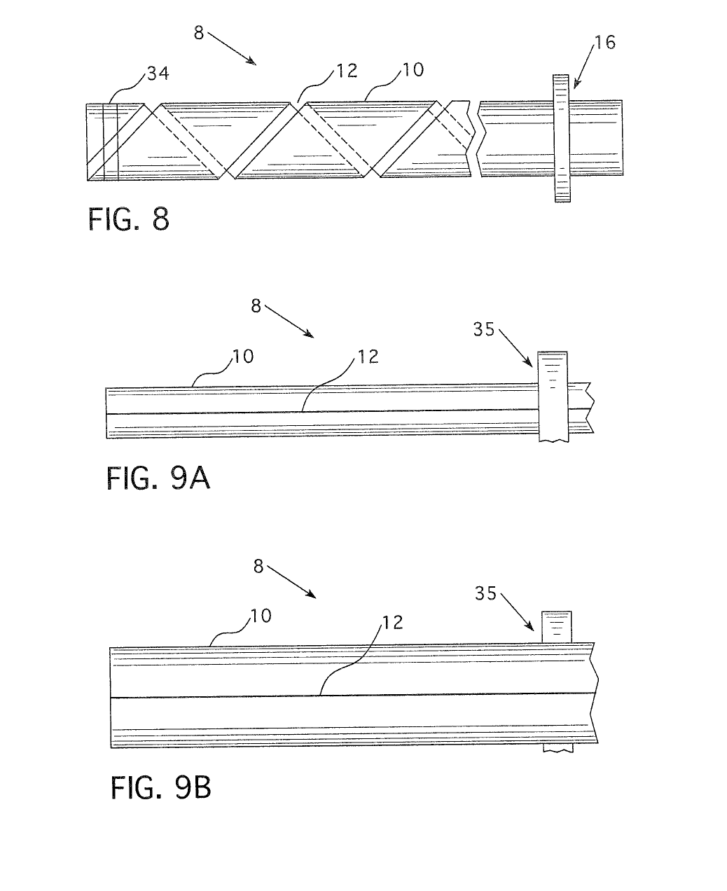

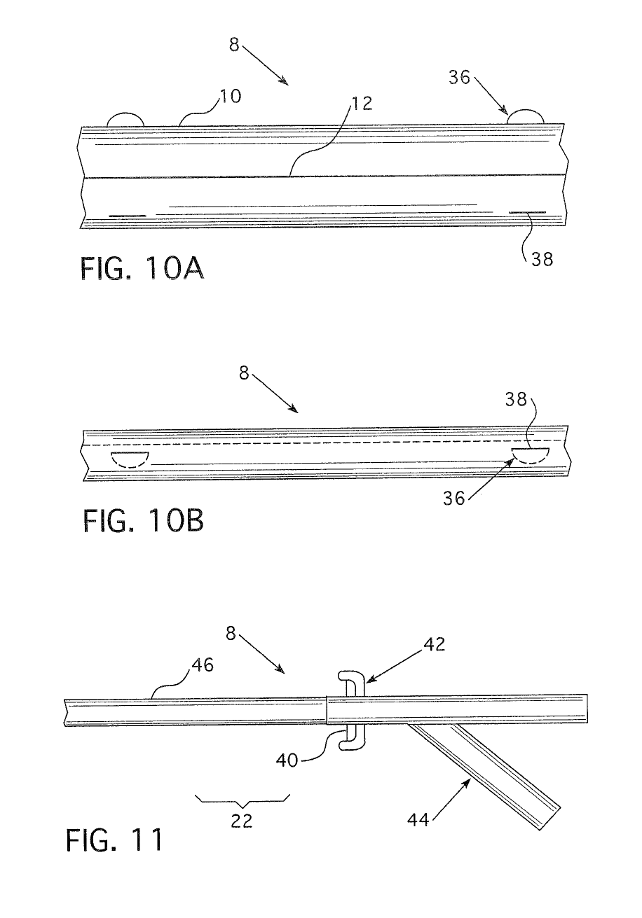

In an alternate embodiment, the tear-away sheath 8 may have a closing feature instead of the handle 14. In an example, the closing feature is a closing band 34 having a hooking or ratchet mechanism with a catch. See FIG. 8. The closing feature may also be an integrated pull-tab 35 fixed at one side of the fault line 12. See FIG. 9A (closed) and 9B (open). After the tear-away sheath 8 containing the balloon 21 has entered the introducer 24 at the appropriate depth via the stop 16 (not shown in FIGS. 9-11), the closing band 34 or the tab 35 is unhooked and pulled away, the tear-away sheath 8 the separates and can be removed. Alternatively, the tear away sheath 8 may be slid back over the strain relief/hub of the catheter 22 thereby splitting the rest of the way. The axial fault line 12 may be either helical or spiral around the tubular element 10 (shown in FIG. 8) or straight (shown, for example, in FIG. 9). In another example, the closing feature may have at least one tab 36 and at least one corresponding slot 38 formed into the tubular element 10. See FIG. 10A. In the closed position, the tab 36 is folded over the fault line 12 and inserted into the slot 38 (FIG. 10B). After introduction of the balloon 21 into the introducer 24, the user pulls the tab 36 from the slot 38, which either opens the tear-away sheath 8 or starts the split at the axial fault line 12. The tear-away sheath 8 can then be removed.

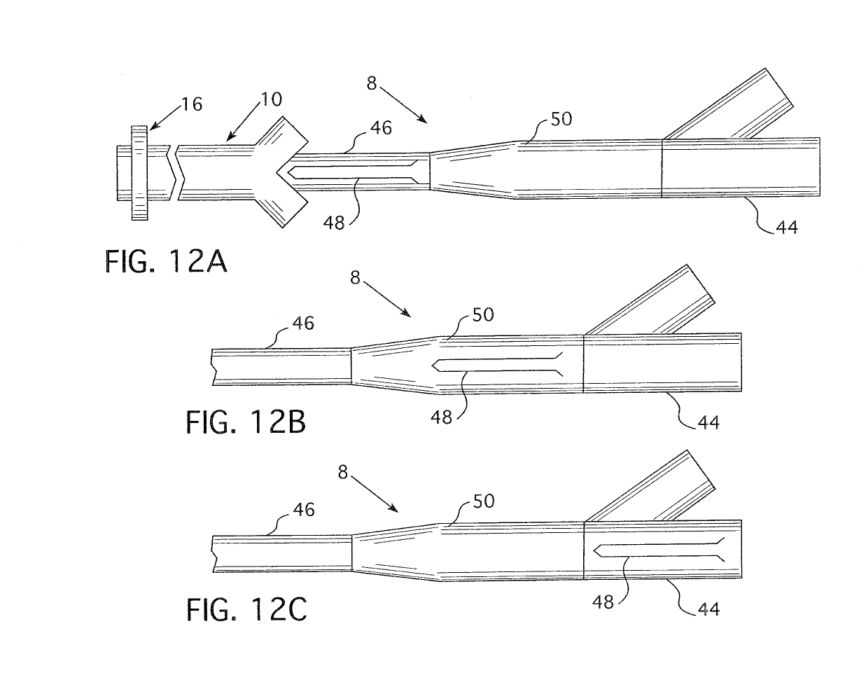

In other embodiments, the tear-away sheath 8 is separated using a cutting device. See FIG. 11. Instead of having a handle 14, an external cutting device as known to those skilled in the art (not shown) can be used to split the tear-away sheath 8. Alternatively, at least one cutting device having a blade 40 and a cover 42 (shown in FIG. 11) may be integrated onto a hub 44 of the balloon catheter 22, the strain relief 50 (not shown in FIG. 11) of the balloon catheter 22, the proximal shaft 46 of the balloon catheter 22, or at any location proximal to the tear-away sheath 8 that does not interfere with use of the device. After introduction of the balloon 21, the user pulls the tear-away sheath 8 back against the blade 40, then grips the split pieces and removes the tear-away sheath 8. In another embodiment, the cutting device is at least one extension 48 integrated into the proximal shaft 46 (FIG. 12A), the strain relief 50 (FIG. 12B), or the hub 44 (FIG. 12C) of a catheter 22, or at any location proximal to the tear-away sheath 8 that does not interfere with use of the device. See FIGS. 12A-C. The extension 48 may be, for example and without limitation, a splitter, a high protrusion wedge, a longitudinal rib, or a different shape or size of the proximal shaft portion 46 (e.g., the outer diameter of the proximal shaft 46 may be greater than the inner diameter of the sheath 8). The tubular element 10 may have at least one perforation or axial fault line 12 corresponding to the extension 48, such that when the tear-away sheath 8 is pulled back it splits apart and can be removed. In addition, pulling the tear-away sheath 8 over the strain relief 50 may split the tear-away sheath 8 for removal.

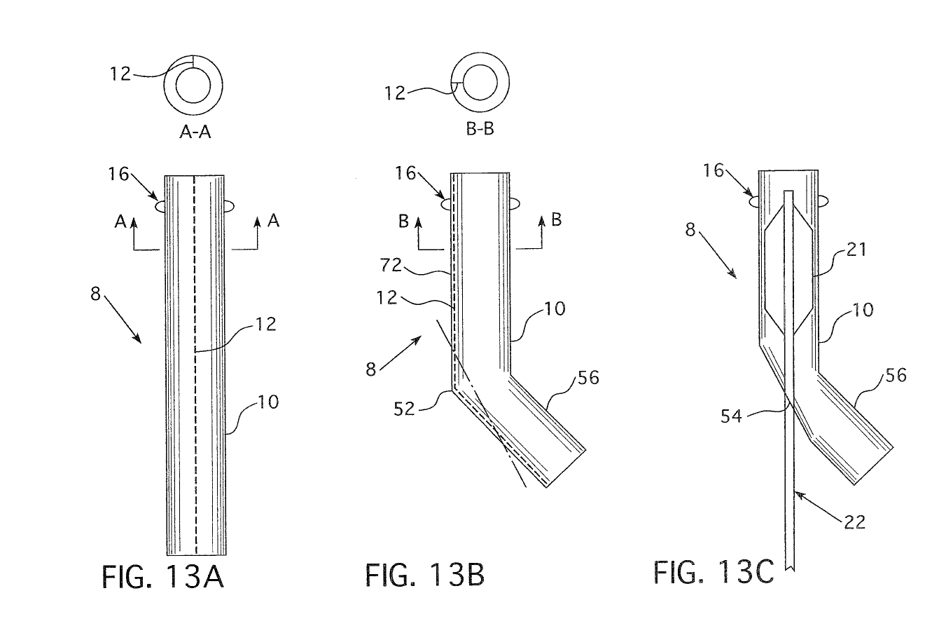

In another embodiment, a tear-away sheath 8 is disclosed having the tubular element 10, at least one axial fault line 12, such as a slit, and the stop 16. To manufacture this embodiment, after the tubular element 10 is formed (FIG. 13A), the proximal end portion is bent at an axial fault line 12 near the proximal end of the balloon 21 (not shown) at a distance to allow protection of the balloon 21 and its coating when it is positioned in the tubular element 10 (FIG. 13B). The elbow end 52 is cut off forming an opening 54 and a wing 56 (FIG. 13C). A coated balloon catheter 22 is inserted into the opening 54 for subsequent distribution and use. During a therapeutic procedure, after insertion of the balloon 21 into the introducer 24 (not shown), the user pulls the tear-away sheath 8 back, holding the wing 56, and removes the tear-away sheath 8 by pulling the wing 56 away from the balloon catheter 22. For example, the balloon catheter 22 slips through the axial fault line 12, e.g. a slit, as the tear-away sheath 8 is pulled away from the shaft of the balloon catheter 22.

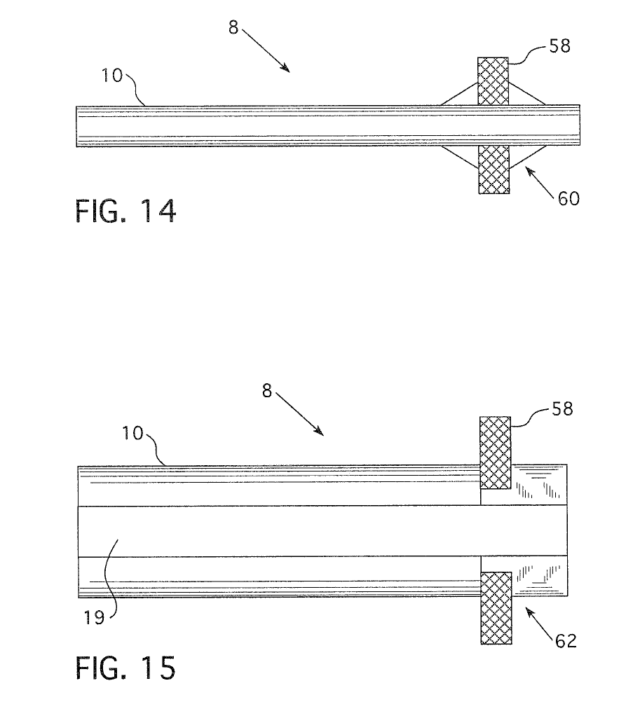

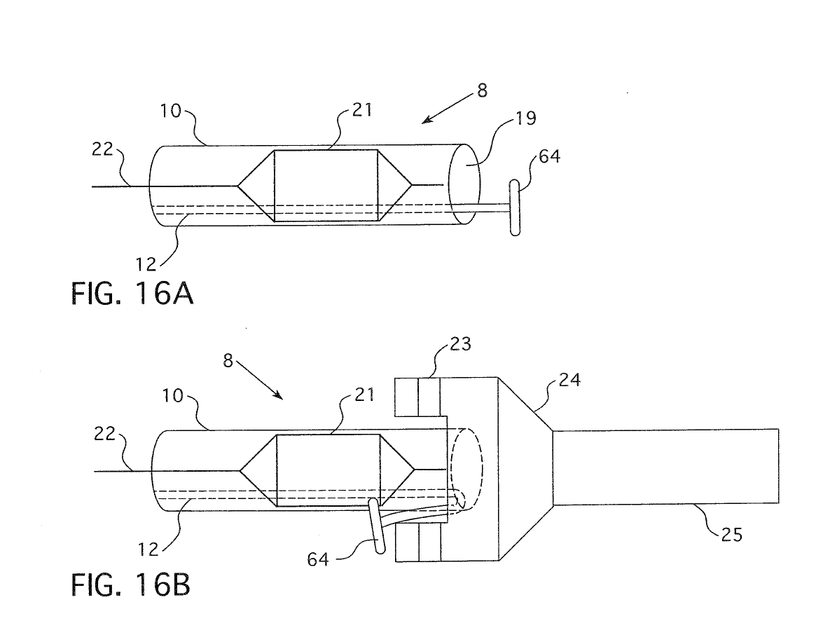

In another embodiment, a tear-away sheath 8 is disclosed having the tubular element 10, at least one axial fault line 12 (not shown), a ring 58, and a retaining element. See FIGS. 14 and 15. In an example, the ring 58 may be a press-fit O-ring. The ring 58 may be split or may have at least one axial fault line 12 (not shown), which may be aligned with the at least one axial fault line 12 of the tubular element 10. The retaining element holds the ring 58 in place to form a physical stop, similar to the physical stop 16 described herein above. In an example, the retaining element may be molded into the tubular element 10, such as at least one lip 60 (shown in FIG. 14) or an indentation in the tubular element 10 (not shown). In another example, the retaining element may be an end cap 62 that is connected to the distal end of the tubular element 10 by ultrasonic welding, infrared (IR) welding, adhesive or chemical bonding, laser welding, and the like. The end cap 62 has the same diameter as lumen 19 thereby making a smooth inner surface while at the same time locking the ring 58 into place. See FIG. 15.

In another embodiment, a tear-away sheath 8 is disclosed having a tubular element 10, at least one axial fault line 12, and an integrated stop-tab 64. See FIG. 16A. The integrated stop-tab 64 may be aligned with the axial fault line 12. The integrated stop-tab 64 may be flexible and extend beyond the tubular element 10 as shown in FIGS. 16A and 16B. For purposes of illustration only, the coated balloon 21 appears without folds in FIGS. 16A and B in order to demonstrate the relation of the tear-away sheath 8, the introducer 24, and the stop-tab 64. Those of skill in the art will understand that coated balloon 21 in its folded state inside of the tear-away sheath 8 has a profile that is not much larger than that of the catheter 22. In an example, a tear-away sheath 8 protecting a coated balloon 21 attached to the balloon catheter 22 is inserted into an introducer 24 and through the introducer flexible valve 23 until the stop-tab 64 contacts the cap of the introducer 24. See FIG. 16B. The balloon 21 may then be advanced into the vasculature while stop-tab 64 prevents movement of the tear-away sheath 8. The tear-away sheath 8 may be removed by pulling the stop-tab 64, for example, along an axial fault line 12. Alternatively, the stop-tab 64 may not be a separate tubular element as depicted in FIGS. 16A and B, but rather is attached to the distal end of the tubular element 10. The attachment may be by a means herein described, including for example but not limited to molding, welding, adhesives, and the like, and ensures maximum lumen 19 space for the coated balloon 21. Those of skill in the art will appreciate the additional open space within the lumen 19 for transfer and movement of the coated balloon 21.

Other embodiments of the present disclosure relate to methods of using embodiments of the disclosed tear-away sheath 8, particularly with a balloon catheter 22. In an example, after a therapeutic coating has been applied to the catheter balloon 21 and allowed to partially dry, the tear-away sheath 8 is placed on the balloon 21 and positioned so that the distal tip of the sheath 8 is aligned with the distal tip of the catheter 22. The catheter 22 with its tear-away sheath 8 is then placed in a tray and the assembly sealed, for example, in a Mylar® film and/or Tyvek® sheet pouch for sterilization, transport, and storage. Once in a clinical setting, the balloon catheter 22 is removed from the tray and the balloon 21 is de-aerated with the tear-away sheath 8 in place. The distal end of the tear-away sheath 8 is then placed into the cap of the hemostasis valve introducer 24 until the stop 16 of the tear-away sheath 8 touches the face of the cap of the introducer 24. See FIG. 3. One hand is used to secure the handle 14 of the tear-away sheath 8 to keep it inserted in and axially aligned with the introducer 24. The other hand is used to feed the balloon 21 through the introducer 24 by pushing on the shaft of the catheter 22 proximal to the sheath 8 in small axial increments to minimize potential kinking of the shaft. Once the balloon 21 is through the hemostasis valve 23, the tear-away sheath 8 is retracted from the introducer 24, positioned more proximally along the shaft of the catheter 22, and removed. For example, a tear-away sheath 8 handle 14 is then split by breaking apart the tabs or wings of the handle 14 and then pulling the tabs away from each other in a continuous motion. The tear-away sheath 8 splits along its fault line(s) 12 until the tear-away sheath 8 has completely separated and can be removed from the shaft of the catheter 22. This method may be used with any of the embodiments disclosed herein.

The disclosed tear-away sheath 8 allows for installation and removal of the sheath 8 during use without affecting a therapeutic coating. The disclosed embodiments allow the tear-away sheath 8 to remain on a balloon catheter 22 or other device until it has been introduced into the vascular system of a patient. See FIG. 3. As such, contact of any therapeutic coating by lab personnel and with the flexible valve 23 of introducers 24 is minimized. The exposure of medical personnel to therapeutic particles released prematurely from the balloon 21 or other device outside the patient is reduced. Therapeutic delivery to the target site may also be maximized.

The disclosed embodiments may be arranged in any combination to provide a tear-away sheath 8 for use with medical devices, particularly balloon catheters 22. In addition, the physical stop 16 on the tubular element 10 may not be required. For example, the inside taper (not shown) of the introducer 24 may provide a “stop” for the tear-away sheath 8. As such, a tear-away sheath 8 without a physical stop 16 is also embodied herein. See FIG. 1A.

### EXAMPLES

The following discussion illustrates non-limiting examples of embodiments of the present disclosure.

A tear-away sheath was made to fit a 3.5×120 mm balloon on a balloon catheter using the overall design shown in FIG. 1B. The tear-away sheath was made entirely of high density polyethylene (HDPE) to provide a desired stiffness, good tearing characteristics, reasonable lubricity, and excellent bondability between the tubular element, the distal stop, and the handle. The sheath was 5.4 inches in length from the most distal part of the handle to the most distal point of the tubular element. The tubular element was 0.053×0.093 inches in cross-section. A stop with an outer diameter of 0.200 inches was positioned 0.400 inches from the distal end of the tubular element. Two axial fault lines were placed in the tubular element using a sharp blade slid along the length of the tubular element using a fixture. The axial fault lines were diametrically opposite one another and passed through the stop. The handle had a maximum tab width of 1.3 inches with an outer hub diameter at the tubular element interface of 0.1875 inches.

A tear-away sheath was designed similar to the embodiment illustrated in FIG. 1B to fit a 6 mm×40 mm percutaneous transluminal angioplasty balloon catheter for use in a femoral popliteal drug coated balloon catheter introduction into a valve. The tear-away sheath was made entirely of PTFE to provide the desired stiffness, lubricity, and tearing characteristics for the axial fault lines. The tubular element of the sheath was plasma treated prior to chemical adhesive bonding of the stop. The tear-away sheath was 3 inches in length from the most distal part of the handle to the most distal part of the tubular element. The tubular element had a cross-section of 0.075×0.101 inches. The stop had a maximum outer diameter of 0.2 inches tapering in the distal direction to 0.101 inches. The tapering of the stop began 0.4 inches from the distal end of the tear-away sheath. The handle had a maximum tab width of 1.47 inches and an outer hub diameter at the tubular element interface of 0.25 inches.

A tear-away sheath for use with a 7×120 mm coated balloon catheter was designed similar to the embodiment illustrated in FIG. 1A. The tubular element was made from high-density polyethylene (HDPE), Ineos K44-24-122, a biocompatible material which is lubricious and strong enough to provide the desired axial support. The tubular element was extruded to dimensions of a 0.081 inch inner diameter, a 0.103 inch outer diameter, and a length of 6.5 inches. Two axial fault lines were extruded into the outside of the tubular element wall diametrically opposite one another at a depth of 0.008 inches and traversed the entire length of the tubular element. The proximal end of the tubular element was flared slightly before an HDPE handle was insert molded onto the tubular element to create an integral bond between the tubular element and the handle. The internal feature of the handle had a distal tapered cone matching the inner diameter of the tubular element and flared proximally at a 45 degree angle until it reached the proximal flat of the handle. Two indentations or notches were molded into the handle which aligned with the two axial fault lines of the tubular element allowing the handle to be broken apart to form two tabs with which to separate the sheath axially along the fault lines.

A tear-away sheath for use with a 3.5×120 mm coated balloon catheter was designed similar to FIG. 13. The tubular element was made from HDPE, Ineos K44-24-122, a biocompatible material which is lubricious and strong enough to provide the desired axial support. The tubular element was extruded to dimensions of a 0.043 inch inner diameter, a 0.071 inch outer diameter, and a length of 6.5 inches. An axial fault line was created by slitting the tubular element with a razor blade supported in a fixture such that the slit completely transected the entire wall thickness. The axial fault line ran along the outside of the tubular element for the entire length. A cut was placed in the proximal end of the tube and the section proximal to the cut was bent as shown in FIGS. 13B and 13C. A flat tab, 0.375×1.00 inches was molded onto the angled wing. The tab was used to pull in a plane comprising the tab and the axial fault line in a direction opposite the axial fault line causing the slit of the axial fault line to open and allow the catheter shaft to pass through the slit thereby releasing the tear-away sheath from the catheter.

The present disclosure has been described with reference to specific details of particular embodiments thereof. It is not intended that such details be regarded as limitations upon the scope of the disclosure except insofar as and to the extent that they are included in the accompanying claims.

What is claimed is:

1. A tear-away sheath comprising a tubular element having a proximal end, a distal end, at least one axial fault line, and a stop.

2. The tear-away sheath of claim 1, wherein the tubular element and the stop are formed from a plastic selected from the group consisting of Teflon® polymers, polyethylenes, polypropolyene, nylons, polyester, and polyvinylchloride.

3. The tear-away sheath of claim 1, further comprising a handle at the proximal end.

4. The tear-away sheath of claim 3, wherein the handle is formed from a plastic selected from the group consisting of polycarbonates, nylons, acrylics, Teflon® polymers, polyethylenes, polypropolyene, polyester, and polyvinylchloride.

5. The tear-away sheath of claim 3, wherein the handle has a plurality of wings with at least one notch aligned with the at least one axial fault line.

6. The tear-away sheath of claim 1, wherein the at least one axial fault line is at least one of a slit, a slot, a notch, a thinner section extruded into the tubular element, a score line, a perforated zipper, or a characteristic of a plastic used to form the tubular element.

7. The tear-away sheath of claim 1 having two axial fault lines on diametrically opposite sides.

8. The tear-away sheath of claim 1, wherein the stop has at least one axial fault line aligned with the at least one axial fault line of the tubular element.

9. The tear-away sheath of claim 1, further comprising a closing band at the proximal end.

10. The tear-away sheath of claim 1, further comprising a pull-tab at the proximal end.

11. The tear-away sheath of claim 1, wherein the at least one axial fault line is helical.

12. The tear-away sheath of claim 1, further comprising at least one tab and at least one corresponding slot at the proximal end.

13. The tear-away sheath of claim 1, wherein the tubular element has an opening and at least one of a wing or a tab at the proximal end.

14. The tear-away sheath of claim 1, wherein the stop comprises a ring and a retaining feature.

15. The tear-away sheath of claim 1, wherein the stop comprises a stop-tab.

16. The tear-away sheath of claim 1, wherein the stop is located near the distal end of the tubular element.

17. A balloon catheter comprising a shaft having a distal end, a proximal end, and at least one lumen, a balloon on the distal end of the shaft, a strain relief and a hub at the proximal end of the shaft, and a tear-away sheath over at least the balloon, wherein the tear-away sheath comprises a tubular element having a proximal end, a distal end, at least one axial fault line, and a stop.

18. The balloon catheter of claim 17, wherein the tear-away sheath further comprises a handle at the proximal end.

19. The balloon catheter of claim 17, wherein the stop of the tear-away sheath has at least one axial fault line aligned with the at least one axial fault line of the tubular element.

20. The balloon catheter of claim 17, wherein the tubular element and the stop of the tear-away sheath are formed from a plastic selected from the group consisting of Teflon® polymers, polyethylenes, polypropolyene, nylon, polyester, and polyvinylchloride.
